(19) United States
(12) Patent Application Publication
(10) Pub. No.: US 2023/0409684 A1
(43) Pub. Date: Dec. 21, 2023

(54) BIOMETRIC AUTHENTICATION SYSTEM, BIOMETRIC AUTHENTICATION METHOD, AND COMPUTER PROGRAM

(71) Applicant: NEC Corporation, Minat-ku, Tokyo (JP)

(72) Inventor: Hiroshi SHIMAMOTO, Tokyo (JP)

(73) Assignee: NEC Corporation, Minat-ku, Tokyo (JP)

(21) Appl. No.: 18/037,530

(22) PCT Filed: Nov. 27, 2020
(86) PCT No.: PCT/JP2020/044261

§ 371 Date: May 17, 2023

(51) Int. Cl. G06F 21/32; G10K 15/04

(52) U.S. Cl. CPC G06F 21/32; G10K 15/04

(57) ABSTRACT

A biometric authentication system includes: an acquisition unit that obtains a feature quantity of a living body; a generation unit that divides the feature quantity and generates a plurality of divided feature quantities; and a collation unit that performs a collation process of the living body by using the plurality of divided feature quantities. According to such a biometric authentication, it is possible to improve the accuracy of the collation process.

### TECHNICAL FIELD

This disclosure relates to a biometric authentication system, a biometric authentication method, and a computer program that perform biometric authentication.

### BACKGROUND ART

A known system of this type combines a plurality of feature quantities when the feature quantities of a living body are collated. For example, Patent Literature 1 discloses a technique/technology of performing the biometric authentication by obtaining a feature quantity used in collation of a feature point type and a feature quantity used in collation of a pattern matching type, from a fingerprint image of a finger.

### CITATION LIST

### Patent Literature

Patent Literature 1: JP6244996B

### SUMMARY

### Technical Problem

This disclosure aims to improve the related technique/technology described above.

### Solution to Problem

A biometric authentication system according to an example aspect of this disclosure includes: an acquisition unit that obtains a feature quantity of a living body; a generation unit that divides the feature quantity and generates a plurality of divided feature quantities; and a collation unit that performs a collation process of the living body by using the plurality of divided feature quantities.

A biometric authentication method according to an example aspect of this disclosure includes: obtaining a feature quantity of a living body; dividing the feature quantity and generating a plurality of divided feature quantities; and performing a collation process of the living body by using the plurality of divided feature quantities.

A computer program according to an example aspect of this disclosure operates a computer: to obtain a feature quantity of a living body; to divide the feature quantity and generate a plurality of divided feature quantities; and to perform a collation process of the living body by using the plurality of divided feature quantities.

### BRIEF DESCRIPTION OF DRAWINGS

FIG. 1 is a block diagram illustrating a hardware configuration of a biometric authentication system according to a first example embodiment.

FIG. 2 is a block diagram illustrating a functional configuration of the biometric authentication system according to the first example embodiment.

FIG. 3 is a flowchart illustrating a flow of operation of the biometric authentication system according to the first example embodiment.

FIG. 4 is a block diagram illustrating a functional configuration of a biometric authentication system according to a second example embodiment.

FIG. 5 is a flowchart illustrating a flow of operation of the biometric authentication system according to the second example embodiment.

FIG. 6 is a flowchart illustrating a flow of operation of a biometric authentication system according to a third example embodiment.

FIG. 7 is a conceptual diagram illustrating an example of dividing a feature quantity by the biometric authentication system according to the third example embodiment.

FIG. 8 is a flowchart illustrating a flow of operation of a biometric authentication system according to a fourth example embodiment.

FIG. 9 is a conceptual diagram illustrating an example of dividing the feature quantity by the biometric authentication system according to the fourth example embodiment.

FIG. 10 is a flowchart illustrating a flow of operation of a biometric authentication system according to a fifth example embodiment.

FIG. 11 is a score matrix illustrating an example of a matching score calculated in a collation process.

FIG. 12 is a map for visualizing the matching score.

FIG. 13 is a conceptual diagram (version 1) illustrating an example of the collation process by the biometric authentication system according to the fifth example embodiment.

FIG. 14 is a conceptual diagram (version 2) illustrating an example of the collation process by the biometric authentication system according to the fifth example embodiment.

FIG. 15 is a block diagram illustrating a functional configuration of a biometric authentication system according to a sixth example embodiment.

FIG. 16 is a flowchart illustrating a flow of operation of the biometric authentication system according to the sixth example embodiment.

FIG. 17 is a block diagram illustrating a functional configuration of a biometric authentication system according to a seventh example embodiment.

FIG. 18 is a flowchart illustrating a flow of operation of the biometric authentication system according to the seventh example embodiment.

### DESCRIPTION OF EXAMPLE EMBODIMENTS

Hereinafter, a biometric authentication system, a biometric authentication method, and a computer program according to example embodiments will be described with reference to the drawings.

### First Example Embodiment

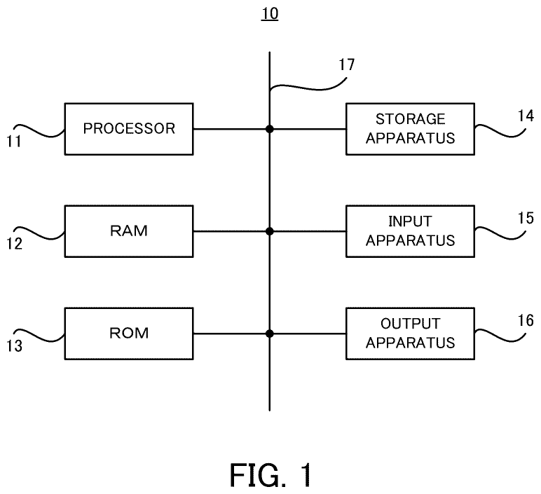

A biometric authentication system according to a first example embodiment will be described with reference to FIG. 1 to FIG. 3.

### (Hardware Configuration)

First, with reference to FIG. 1, a hardware configuration of the biometric authentication system according to the first example embodiment will be described. FIG. 1 is a block diagram illustrating the hardware configuration of the biometric authentication system according to the first example embodiment.

As illustrated in FIG. 1, a biometric authentication system 10 according to the first example embodiment includes a processor 11, a RAM (Random Access Memory) 12, a ROM (Read Only Memory) 13, and a storage apparatus 14. The biometric authentication system 10 may further include an input apparatus 15 and an output apparatus 16. The processor 11, the RAM 12, the ROM 13, the storage apparatus 14, the input apparatus 15, and the output apparatus 16 are connected through a data bus 17.

The processor 11 reads a computer program. For example, the processor 11 is configured to read a computer program stored by at least one of the RAM 12, the ROM 13 and the storage apparatus 14. Alternatively, the processor 11 may read a computer program stored in a computer readable recording medium by using a not-illustrated recording medium reader apparatus. The processor 11 may obtain (i.e., read) a computer program from a not-illustrated apparatus disposed outside the biometric authentication system 10, through a network interface. The processor 11 controls the RAM 12, the storage apparatus 14, the input apparatus 15, and the output apparatus 16 by executing the read computer program. Especially in the example embodiment, when the processor 11 executes the read computer program, a functional block for performing various processes related to a feature quantity of a living body is realized or implemented in the processor 11. An example of the processor 11 includes a CPU (Central Processing Unit), a GPU (Graphics Processing Unit), a FPGA (field-programmable gate array), a DSP (Demand-Side Platform), and an ASIC (Application Specific Integrated Circuit). The processor 11 may use one of the above examples, or may use a plurality of them in parallel.

The RAM 12 temporarily stores the computer program to be executed by the processor 11. The RAM 12 temporarily stores the data that is temporarily used by the processor 11 when the processor 11 executes the computer program. The RAM 12 may be, for example, a D-RAM (Dynamic RAM).

The ROM 13 stores the computer program to be executed by the processor 11. The ROM 13 may otherwise store fixed data. The ROM 13 may be, for example, a P-ROM (Programmable ROM).

The storage apparatus 14 stores the data that is stored for a long term by the biometric authentication system 10. The storage apparatus 14 may operate as a temporary storage apparatus of the processor 11. The storage apparatus 14 may include, for example, at least one of a hard disk apparatus, a magneto-optical disk apparatus, an SSD (Solid State Drive), and a disk array apparatus.

The input apparatus 15 is an apparatus that receives an input instruction from a user of the biometric authentication system 10. The input apparatus 15 may include, for example, at least one of a keyboard, a mouse, and a touch panel. The input apparatus 15 may be a dedicated controller (operation terminal). The input apparatus 15 may also include a terminal owned by the user (e.g., a smartphone or a tablet terminal). The input apparatus 15 may be an apparatus that allows an audio input including a microphone, for example.

The output apparatus 16 is an apparatus that outputs information about the biometric authentication system 10 to the outside. For example, the output apparatus 16 may be a display apparatus (e.g., a display) that is configured to display the information about the biometric authentication system 10. The display apparatus here may be a TV monitor, a personal computer monitor, a smartphone monitor, a tablet terminal monitor, or another portable terminal monitor. The display apparatus may be a large monitor or a digital signage installed in various facilities such as stores. The output apparatus 16 may be an apparatus that outputs the information in a format other than an image. For example, the output apparatus 16 may be a speaker that audio-outputs the information about the biometric authentication system 10.

### (Functional Configuration)

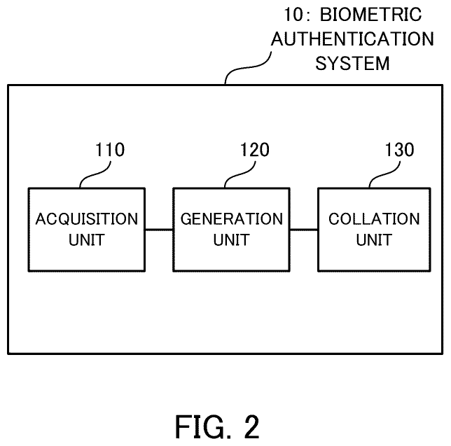

Next, with reference to FIG. 2, a functional configuration of the biometric authentication system 10 according to the first example embodiment will be described. FIG. 2 is a block diagram illustrating the functional configuration of the biometric authentication system according to the first example embodiment.

As illustrated in FIG. 2, the biometric authentication system 10 according to the first example embodiment includes, as processing blocks for realizing its functions, an acquisition unit 110, a generation unit 120, and a collation unit 130. Each of the acquisition unit 110, the generation unit 120, and the collation unit 130 may be realized or implemented by the processor 11 (see FIG. 1).

The acquisition unit 110 is configured to obtain a feature quantity of the living body. The feature quantity here is a parameter used for a collation process in biometric authentication, and can be obtained from a face (eyes, ears, a nose, a mouth), skin, or the like of the living body, for example. A detailed description of a specific method of obtaining the feature quantity will be omitted, because the existing techniques/technologies can be adopted as appropriate. The feature quantity obtained by the acquisition unit 110 is configured to be outputted to the generation unit 120.

The generation unit 120 divides the feature quantity of the living body obtained by the acquisition unit 110 and generates a plurality of divided feature quantities. The generation unit 120 generates the divided feature quantities by separately dividing a plurality of parameters included in the feature quantity, for example. Therefore, it is preferable that the feature quantity obtained by the acquisition unit 110 includes the plurality of parameters (e.g., data of a plurality of dimensions). Alternatively, the generation unit 120 may convert the feature quantity obtained by the acquisition unit 110 into a divisible condition, before dividing it. A specific method of generating the divided feature quantities will be described in detail in another example embodiment described later. The plurality of divided feature quantities generated by the generation unit 120 are configured to be outputted to the collation unit 130.

The collation unit 130 performs the collation process of the living body by using the plurality of divided feature quantities generated by the generation unit 120. Specifically, the collation unit 130 performs a collation for each of the plurality of divided feature quantities. The collation unit 130 may include a database that stores a feature quantity (i.e., a registered feature quantity of the living body) that is compared and collated with the obtained divided feature quantities. The collation unit 130 may compare the obtained divided feature quantities and the registered feature quantity, and may output a collation result based on their degree of matching (similarity) or the like, for example. The collation unit 130 may have a threshold for determining whether the collation is successful or failed from a comparison result. A specific example of the collation process will be described in detail in another example embodiment described later.

### (Flow of Operation)

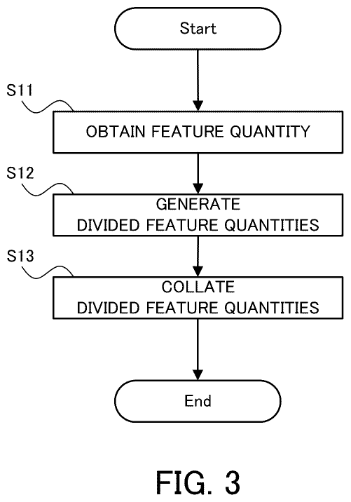

Next, with reference to FIG. 3, a flow of operation of the biometric authentication system according to the first example embodiment will be described. FIG. 3 is a flowchart illustrating the flow of the operation of the biometric authentication system according to the first example embodiment.

As illustrated in FIG. 3, when the operation of the biometric authentication system 10 according to the first example embodiment is started, first, the acquisition unit 110 obtains the feature quantity of the living body (step S11). Subsequently, the generation unit 120 divides the feature quantity and generates a plurality of divided feature quantities (step S12). Then, the collation unit 130 performs the collation process of the living body by using the divided feature quantities (step S13). The collation unit 130 may output the result of the collation process (i.e., whether the biometric authentication is successful or failed) to another apparatus.

### (Technical Effect)

Next, a technical effect obtained by the biometric authentication system 10 according to the first example embodiment will be described.

As described in FIG. 1 to FIG. 3, in the biometric authentication system 10 according to the first example embodiment, a plurality of feature quantities are generated by dividing the feature quantity, and the collation process is performed by using the plurality of divided feature quantities. In this way, as compared with when collectively performing the collation without dividing the feature quantity, it is possible to improve a collation accuracy, by an amount of increased parameters to be divided and collated.

### Second Example Embodiment

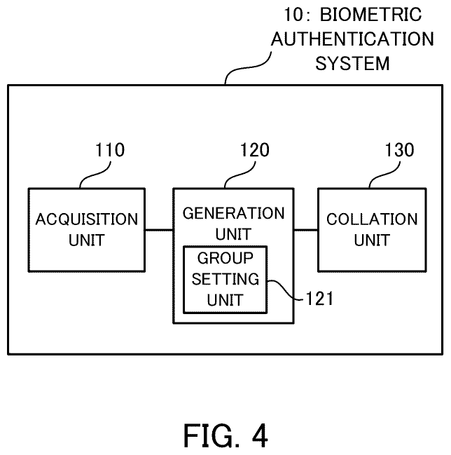

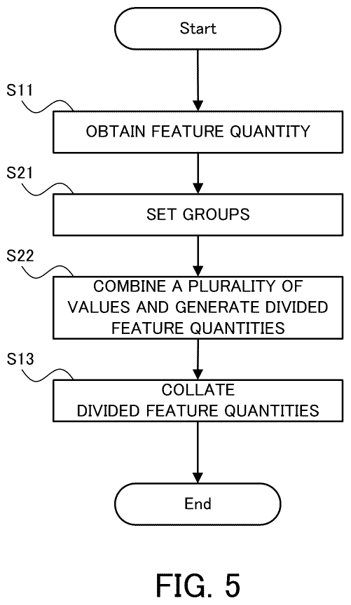

The biometric authentication system 10 according to a second example embodiment will be described with reference to FIG. 4 and FIG. 5. The second example embodiment is partially different from the first example embodiment only in the configuration and operation, may be the same as the first example embodiment in the hardware configuration (see FIG. 1) or the like, for example. For this reason, a part that is different from the first example embodiment will be described in detail below, and a description of the other overlapping parts will be omitted as appropriate.

### (Functional Configuration)

First, with reference to FIG. 4, a functional configuration of the biometric authentication system 10 according to the second example embodiment will be described. FIG. 4 is a block diagram illustrating the functional configuration of the biometric authentication system according to the second example embodiment. In FIG. 4, the same components as those illustrated in FIG. 2 carry the same reference numerals.

As illustrated in FIG. 4, the biometric authentication system 10 according to the second example embodiment includes, as processing blocks for realizing its functions, the acquisition unit 110, the generation unit 120, and the collation unit 130. In particular, the generation unit 120 according to the second example embodiment includes a group setting unit 121. The group setting unit 121 may be realized or implemented by the processor 11 (see FIG. 1).

The group setting unit 121 is configured to set groups for generating divided feature quantities. Specifically, the group setting unit sets a plurality of groups by combining some of a plurality of values included in the feature quantity. Therefore, the generation unit 120 according to the second example embodiment generates the plurality of divided feature quantities, on the basis of the groups set by the group setting unit 121. A specific method of setting the groups will be described in detail in another example embodiment described later.

### (Flow of Operation)

Next, with reference to FIG. 5, a flow of operation of the biometric authentication system according to the second example embodiment will be described. FIG. 5 is a flowchart illustrating the flow of the operation of the biometric authentication system according to the second example embodiment. In FIG. 5, the same steps as those illustrated in FIG. 3 carry the same reference numerals.

As illustrated in FIG. 5, when the operation of the biometric authentication system 10 according to the second example embodiment is started, first, the acquisition unit 110 obtains the feature quantity of the living body (step S11). Subsequently, the group setting unit 121 sets a plurality of groups (step S21). That is, the group setting unit 121 sets combinations each of which includes some of the plurality of values included in the feature quantity. Then, the generation unit 120 divides the feature quantity into a plurality of values, combines some of the plurality of values in accordance with the set groups, and generates a plurality of divided feature quantities (step S22). Then, the collation unit 130 performs the collation process of the living body by using the divided feature quantities (step S13).

### (Technical Effect)

Next, a technical effect obtained by the biometric authentication system 10 according to the second example embodiment will be described.

As described in FIG. 4 and FIG. 5, in the biometric authentication system 10 according to the second example embodiment, a plurality of divided feature quantities are generated by combining a plurality of values. In this way, it is possible to generate the divided feature quantities more flexibly by the combination of the plurality of values. For example, for the plurality of values included in the feature quantity, appropriate values can be combined with each other to generate the divided feature quantities. Furthermore, depending on a combination pattern, it is also possible to generate more divided feature quantities than the number of the plurality of values included in the feature quantity.

### Third Example Embodiment

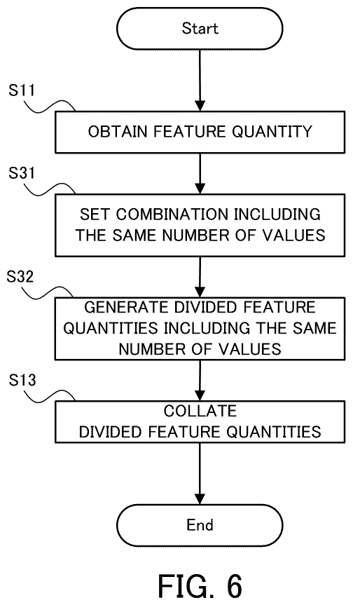

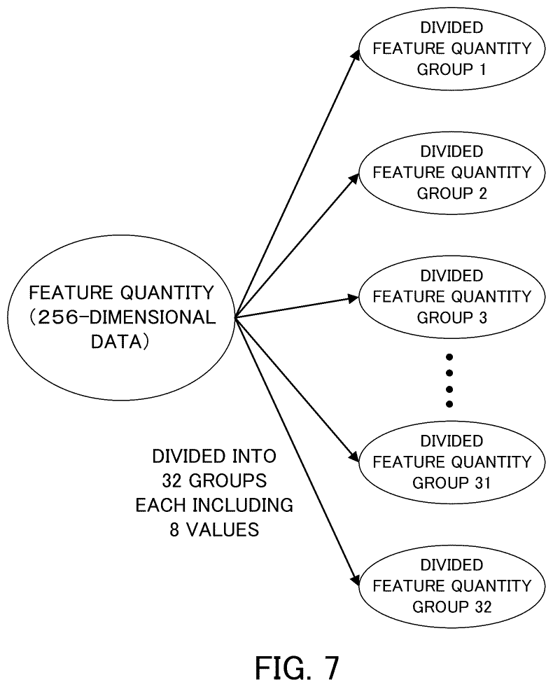

The biometric authentication system 10 according to the third example embodiment will be described with reference to FIG. 6 and FIG. 7. The third example embodiment describes a specific example of the group setting in the second example embodiment described above, and may be the same as the first and second example embodiments in the configuration. For this reason, a part that is different from each of the example embodiment described above will be described in detail below, and a description of the other overlapping parts will be omitted as appropriate.

### (Flow of Operation)

First, with reference to FIG. 6, a flow of operation of the biometric authentication system according to the third example embodiment will be described. FIG. 6 is a flowchart illustrating the flow of the operation of the biometric authentication system according to the third example embodiment. In FIG. 6, the same steps as those illustrated in FIG. 5 carry the same reference numerals.

As illustrated in FIG. 6, when the operation of the biometric authentication system 10 according to the third example embodiment is started, first, the acquisition unit 110 obtains the feature quantity of the living body (step S11). Subsequently, the group setting unit 121 sets a plurality of groups including the same number of values (step S31). Then, the generation unit 120 divides the feature quantity into a plurality of values, and generates a plurality of divided feature quantities such that each group includes the same number of values (step S32). Subsequently, the collation unit 130 performs the collation process of the living body by using the divided feature quantities (step S13).

### (Combination Example)

Next, with reference to FIG. 7, the grouping setting method (i.e., a specific combination example) of the divided feature quantities in the biometric authentication system 10 according to a third example embodiment is described in detail. FIG. 7 is a conceptual diagram illustrating an example of dividing the feature quantity by the biometric authentication system according to the third example embodiment.

As illustrated in FIG. 7, it is assumed that 256-dimensional data (i.e., data including 256 divisible parameters) are obtained as the feature quantity of the living body. In this case, the biometric authentication system 10 according to the third example embodiment may divide the feature quantity into 32 groups each of which includes eight parameters. That is, the group setting unit 121 may set 32 groups each of which includes eight values, and the generation unit 120 may divide the feature quantity in accordance with those groups, may make combinations, and may generate 32 divided feature quantities. The eight values included in each group may be determined to be a combination set in advance. For example, at the time of setting the system, eight values may be selected randomly from 256 values to determine each group.

The grouping described above is merely an example, and it may be divided into 64 groups each of which includes four parameters, or may be divided into 16 groups each of which includes 16 parameters, for example. The number of the groups may be set in advance by the user or the like, or may be set as appropriate on the basis of the type of the obtained feature quantity or the processing content of the collation process that uses the divided feature quantities or the like.

### (Technical Effect)

Next, a technical effect obtained by the biometric authentication system 10 according to the third example embodiment will be described.

As described in FIG. 6 and FIG. 7, in the biometric authentication system 10 according to the third example embodiment, the feature quantity is divided into groups each of which includes the same number of values. In this way, it is possible to easily generate a plurality of divided feature quantities from the feature quantity including a plurality of values. Furthermore, according to a research by the inventors of the present application, it has been found that an appropriate collation result can be obtained when the divided feature quantities each of which includes the same number of values are used. This example will be described in detail in another example embodiment described later.

### Fourth Example Embodiment

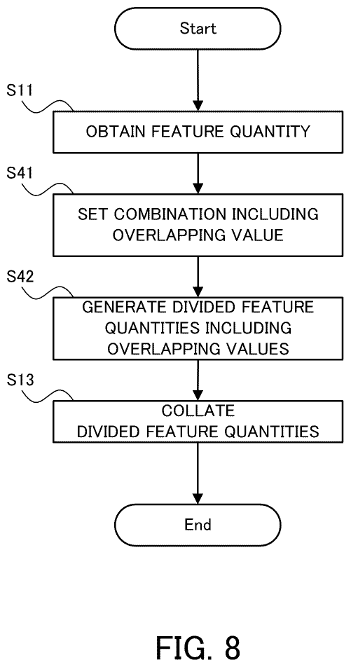

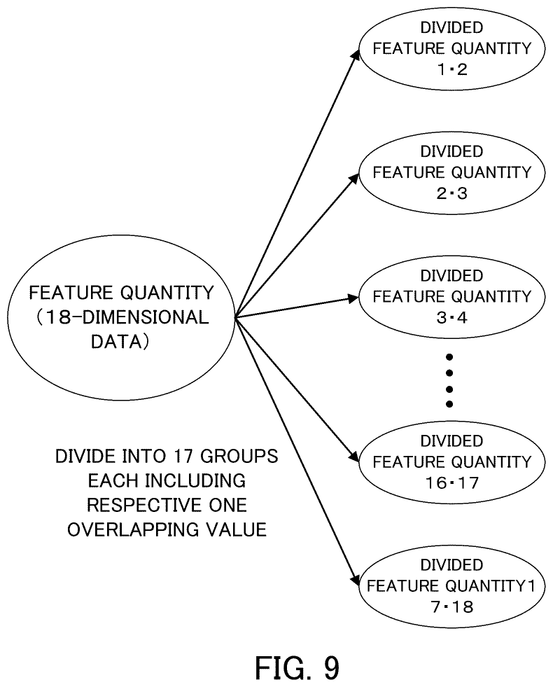

The biometric authentication system 10 according to a fourth example embodiment will be described with reference to FIG. 8 and FIG. 9. The fourth example embodiment describes, as in the third example embodiment, a specific example of the group setting in the second example embodiment, and may be the same as that of the first and second example embodiments in the configuration. For this reason, a part that is different from each of the example embodiment described above will be described in detail below, and a description of the other overlapping parts will be omitted as appropriate.

### (Flow of Operation)

First, with reference to FIG. 8, a flow of operation of the biometric authentication system according to the fourth example embodiment will be described. FIG. 8 is a flowchart illustrating the flow of the operation of the biometric authentication system according to the fourth example embodiment. In FIG. 8, the same steps as those illustrated in FIG. 5 carry the same reference numerals.

As illustrated in FIG. 8, when the operation of the biometric authentication system 10 according to the fourth example embodiment is started, first, the acquisition unit 110 obtains the feature quantity of the living body (step S11). Subsequently, the group setting unit 121 sets a plurality of groups so as to include an overlapping value that is also included in another group (step S41). Then, the generation unit 120 divides the feature quantity into a plurality of values, and generates a plurality of divided feature quantities so as to redundantly include the plurality of values (step S42). Subsequently, the collation unit 130 performs the collation process of the living body by using the divided feature quantities (step S13).

### (Combination Example)

Next, with reference to FIG. 9, the grouping setting method (i.e., a specific combination example) of the divided feature quantities in the biometric authentication system 10 according to the fourth example embodiment will be described in detail. FIG. 9 is a conceptual diagram illustrating an example of dividing the feature quantity by the biometric authentication system according to the fourth example embodiment.

As illustrated in FIG. 9, it is assumed that 18-dimensional data (i.e., data including 18 divisible parameters) are obtained as the feature quantity of the living body. In this case, the biometric authentication system 10 according to the fourth example embodiment may divide the feature quantity into 17 groups each of which includes two values one of which is duplicated in a previous group and the other of which is duplicated in a subsequent group. That is, the group setting unit 121 sets 17 groups each of which includes respective one overlapping value, and the generation unit 120 may divide the feature quantity in accordance with those groups, may make combinations, and may generate 17 divided feature quantities.

For example, when the feature quantity includes values of 1 to 18, the first divided feature quantity includes values of 1 and 2. The second divided feature quantity includes values of 2 and 3. The third divided feature quantity includes values of 3 and 4. The same applies thereafter, and the 16th divided feature quantity includes values of 16 and 17. The 17th divide feature quantity includes values of 17 and 18.

The grouping described above is merely an example, and it may be divided into groups each of which includes two or more parameters in duplicate, for example. In addition, the number of values included in duplicate may vary depending on the group. The number of the overlapping values may be set in advance by the user or the like, or may be set as appropriate on the basis of the type of the obtained feature quantity or the processing content of the collation process that uses the divided feature quantities or the like.

### (Technical Effect)

Next, a technical effect obtained by the biometric authentication system 10 according to the fourth example embodiment will be described.

As described in FIG. 8 and FIG. 9, in the biometric authentication system 10 according to the fourth example embodiment, the feature quantity is divided into groups each of which includes the same number of values. In this way, it is possible to easily generate a plurality of divided feature quantities from the feature quantity including a plurality of values. Furthermore, according to a research by the inventors of the present application, it has been found that an appropriate collation result can be obtained when the divided feature quantities including overlapping values are used. This example will be described in detail in another example embodiment described later.

### Fifth Example Embodiment

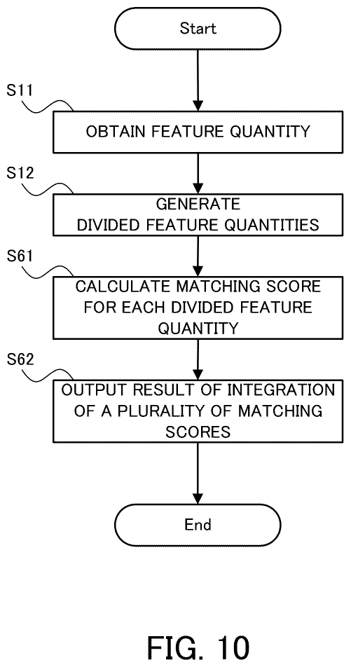

The biometric authentication system 10 according to a fifth example embodiment will be described with reference to FIG. 10 to FIG. 14. The fifth example embodiment is partially different from the first to fourth example embodiments only in the operation, and may be the same as those of the first to fourth example embodiments in other parts. For this reason, a part that is different from each of the example embodiment described above will be described in detail below, and a description of the other overlapping parts will be omitted as appropriate.

### (Flow of Operation)

First, with reference to FIG. 10, a flow of operation of the biometric authentication system according to the fifth example embodiment will be described. FIG. 10 is a flowchart illustrating the flow of the operation of the biometric authentication system according to the fifth example embodiment. In FIG. 10, the same steps as those illustrated in FIG. 3 carry the same reference numerals.

As illustrated in FIG. 10, when the operation of the biometric authentication system 10 according to the fifth example embodiment is started, first, the acquisition unit 110 obtains the feature quantity of the living body (step S11). Subsequently, the generation unit 120 generates a plurality of divided feature quantities by dividing the feature quantity (step S12). Subsequently, the collation unit 130 calculates a plurality of matching scores for each divided feature quantity (step S61). Then, the collation unit 130 outputs a result of integration of a plurality of matching scores (step S62). The integration of the matching scores will be described in detail later.

### (Matching Score)

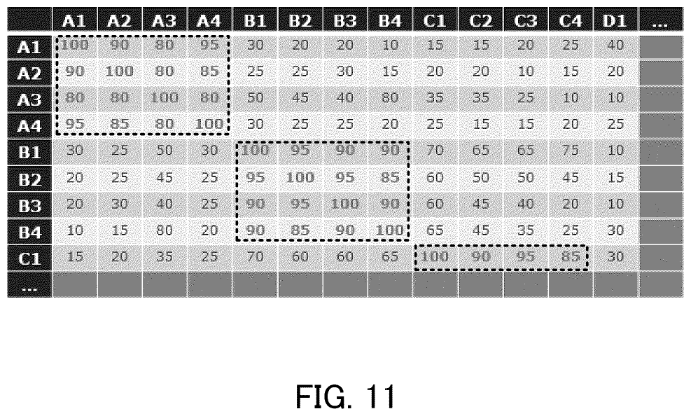

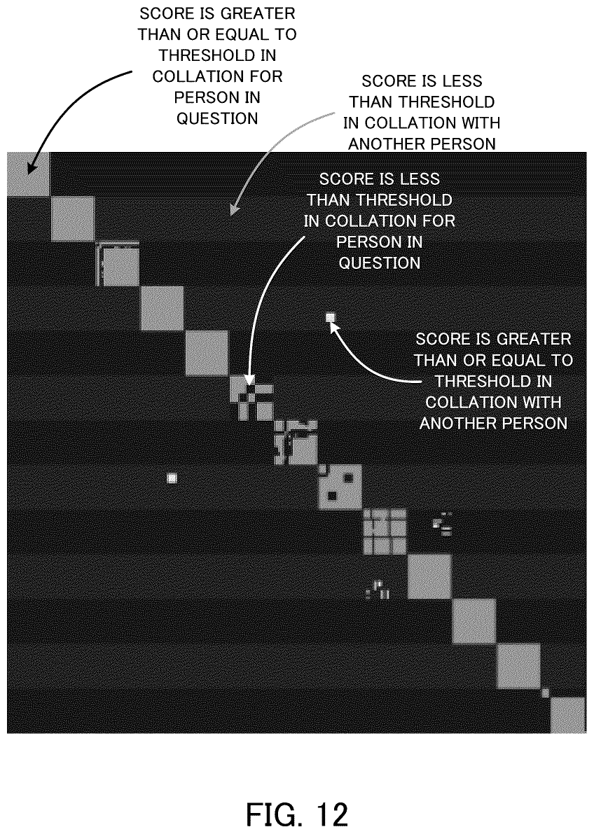

Next, with reference to FIG. 11 and FIG. 12, the matching score used in the biometric authentication system 10 according to the fifth example embodiment will be specifically described. FIG. 11 is a score matrix illustrating an example of the matching score calculated in the collation process. FIG. 12 is a map for visualizing the matching score.

The example illustrated in FIG. 11 shows the matching scores when a plurality of feature quantities obtained from each of living bodies A, B, C, D, and so on (e.g., feature quantities of the living body A is A1, A2, A3 and A4) are collated in a form of round robin, for example. The matching score is calculated on the basis of a degree of similarity (a degree of coincidence) of the feature quantity or the like, for example. The matching score can be calculated by using a cosine similarity, for example. A method of calculating the matching score, however, is not particularly limited, and the matching score may be calculated by employing an existing method, as appropriate.

As is clear from each score, the scores calculated by the collation for a person in question (the score within a frame line in the figure) are higher than the scores calculated by the collation with another person (the score outside the frame line in the figure). For example, the score when collating A1 and A2 is “90”, while the score when collating A1 and B4 is “10”. In the example illustrated in FIG. 10, for example, if a score of “80” is set as a threshold, it is possible to accurately distinguish the person in question and another person. As the threshold, the lowest value of the score for the person in question in the collation result (hereinafter referred to as a person-in-question minimum score) may be set, for example.

The map illustrated in FIG. 12 visualizes the matching scores in FIG. 11 (imaging by changing color). For example, rectangular areas diagonally lined up from a top left to a bottom right of the map correspond to the matching scores calculated by the collation for the person in question, while other areas correspond to the matching scores calculated by the collation with another person. Among the matching scores calculated by the collation for the person in question, those that are greater than or equal to the threshold are indicated in gray, and those that are less than the threshold are indicated in black. Among the matching scores calculated by the collation with another person, those that are greater than or equal to the threshold are indicated in white, and those that are less than the threshold are indicated in black.

In the example illustrated in FIG. 12, there are cases where the matching score calculated by the collation for the person in question is less than the threshold (i.e., when the person in question is denied) and when the matching score calculated by the collation with another person is greater than or equal to the threshold (i.e., another person is allowed). Such an inappropriate result can be reduced by integration of the scores described later.

### (Integration of Scores)

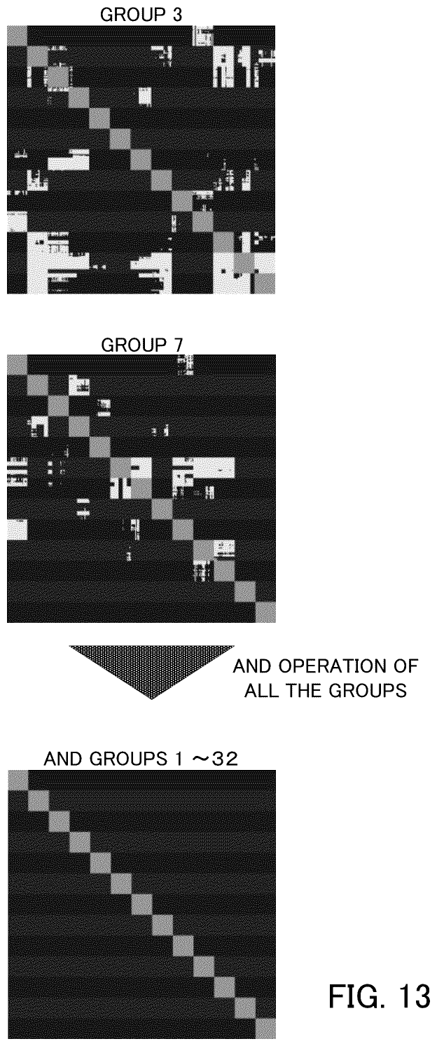

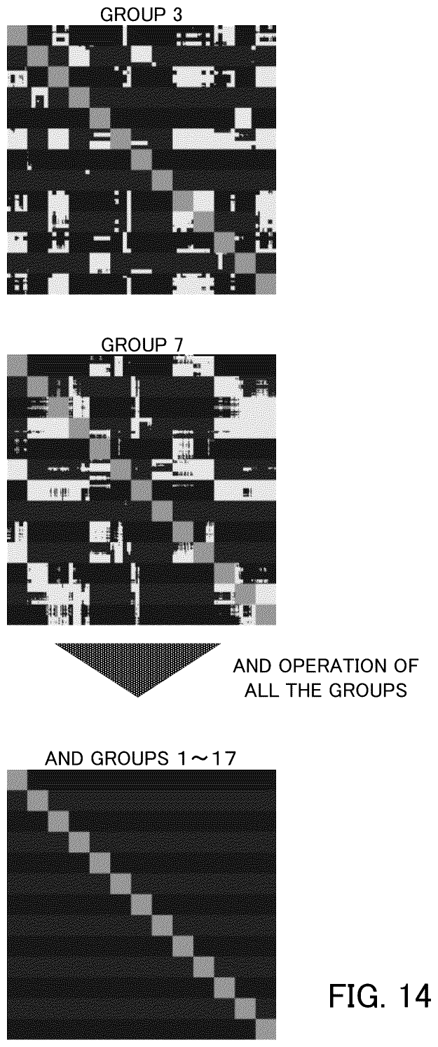

Next, with reference to FIG. 13 and FIG. 14, the integration of the scores will be described. FIG. 13 is a conceptual diagram (version 1) illustrating an example of the collation process by the biometric authentication system according to the fifth example embodiment. FIG. 14 is a conceptual diagram (version 2) illustrating an example of the collation process by the biometric authentication system according to the fifth example embodiment. FIG. 13 and FIG. 14 illustrate maps for visualizing the matching score (for the map, see the description of FIG. 12).

FIG. 13 illustrates a result obtained by collating 32 divided groups that is obtained by dividing the feature quantity that is 256-dimensional data exemplified in the third example embodiment (see FIG. 7), in the form of round robin as illustrated in FIG. 10. For convenience of explanation, not all the 32 groups, but only the collation results of a group 3 and a group 7 are illustrated.

In the example illustrated in FIG. 13, there is partially an area (i.e., a white area in the figure) in which even the matching score of the collation with another person is greater than or equal to the threshold, in both of the collation results of the group 3 and the group 7. Therefore, if the collation results of the group 3 and the group 7 are used independently without change, another person may be allowed in the biometric authentication.

In the biometric authentication system 10 according to the fifth example embodiment, however, a result obtained by integrating the collation results (i.e., the scores) of a plurality of divided feature quantities is outputted. Specifically, an AND operation of the collation results of 32 groups is performed and outputted as one collation result. As can be seen from FIG. 13, in the map where the AND operation is performed, the area in which the matching score of the collation with another person is greater than or equal to the threshold disappears. This is due to the fact that there are relatively many areas in which another person is allowed on a lower side of the map for the group 3, while there are few areas in which another person is allowed on a lower side of the map for the group 7. As described above, the integration of the plurality of scores can reduce or eliminate a likelihood that another person is allowed.

FIG. 14 illustrates a result obtained by collating 17 divided groups that is obtained by dividing the feature quantity that is 18-dimensional data exemplified in the fourth example embodiment (see FIG. 9), in the form of round robin as illustrated in FIG. 10. For convenience of explanation, not all the 17 groups, but only the collation results of a group 3 and a group 7 are illustrated.

In the example illustrated in FIG. 14, there is partially an area (i.e., a white area in the figure) in which even the matching score of the collation with another person is greater than or equal to the threshold, in both of the collation results of the group 3 and the group 7. Therefore, if the collation results of the group 3 and the group 7 are used independently without change, another person may be allowed in the biometric authentication.

In the biometric authentication system 10 according to the fifth example embodiment, however, a result obtained by integrating the collation results (i.e., the scores) of a plurality of divided feature quantities is outputted. Specifically, an AND operation of the collation results of 17 groups is performed and outputted as one collation result. As can be seen from FIG. 14, in the map where the AND operation is performed, the area in which the matching score of the collation with another person is greater than or equal to the threshold disappears. Thus, as in the case illustrated in FIG. 13, the integration of the scores can reduce or eliminate the likelihood that another person is allowed.

In the above example, there is no area in which even the matching score of the collation for the person in question is less than the threshold (i.e., a area in which the person in question is denied). This is because a loose or accommodative threshold is set such that the person in question is not to be denied while leaving some possibility that another person is allowed. The use the divided feature quantities reduces a possibility that there is another person who is allowed in all the groups, even though there may be another person who is allowed in one group. For this reason, the setting of the loose or accommodative threshold may eventually reduce the possibility that another person is allowed. It is thus possible to reduce the possibility that another person is allowed, while increasing a possibility that the person in question is denied.

### (Technical Effect)

Next, a technical effect obtained by the biometric authentication system 10 according to the fifth example embodiment will be described.

As described in FIG. 10 to FIG. 14, in the biometric authentication system 10 according to the fifth example embodiment, the result obtained by integrating the scores calculated for each divided area is outputted. In this way, it is possible to prevent that an inappropriate collation result (e.g., such a result that the person in question is denied, or that another person is allowed) is outputted.

### Sixth Example Embodiment

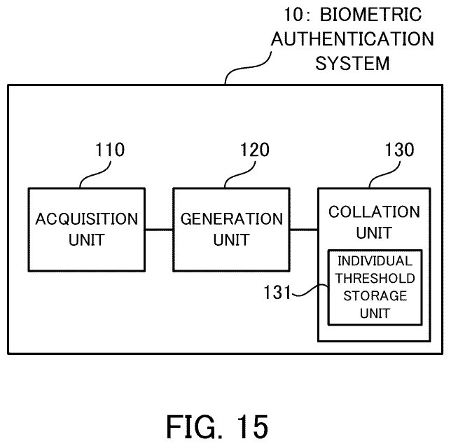

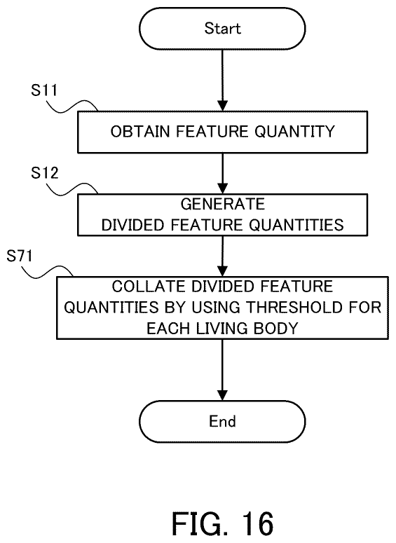

The biometric authentication system 10 according to a sixth example embodiment will be described with reference to FIG. 15 and FIG. 16. The sixth example embodiment is partially different from the first to fifth example embodiments in the configuration and operation, and may be the same as the first to fifth example embodiments in other parts. For this reason, a part that is different from each of the example embodiment described above will be described in detail below, and a description of the other overlapping parts will be omitted as appropriate.

### (Functional Configuration)

First, with reference to FIG. 15, a functional configuration of the biometric authentication system 10 according to the sixth example embodiment will be described. FIG. 15 is a block diagram illustrating the functional configuration of the biometric authentication system according to the sixth example embodiment. In FIG. 15, the same components as those illustrated in FIG. 2 carry the same reference numerals.

As illustrated in FIG. 15, the biometric authentication system 10 according to the sixth example embodiment includes, as processing blocks for realizing its functions, the acquisition unit 110, the generation unit 120, and the collation unit 130. In particular, the collation unit 130 according to the second example embodiment includes an individual threshold storage unit 131. The individual threshold storage unit 131 may be realized or implemented by the storage apparatus 14 (see FIG. 1).

The individual threshold storage unit 131 is configured to be store a threshold used in the collation process (e.g., the threshold related to the matching score described in the sixth example embodiment). The individual threshold storage unit 131 stores the threshold for each living body. The individual threshold storage unit 131 stores, as separate thresholds, a threshold used for the collation of the living body A, the threshold used for the collation of the living body B, and the threshold used for the collation of the living body C, for example. The threshold stored by the individual threshold storage unit 131 may be the value of the person-in-question minimum score described in the fifth example embodiment, for example. The threshold stored in the individual threshold storage unit 131 may be read as appropriate by the collation unit 130.

### (Flow of Operation)

Next, with reference to FIG. 16, a flow of operation of the biometric authentication system according to the sixth example embodiment will be described. FIG. 16 is a flowchart illustrating the flow of the operation of the biometric authentication system according to the sixth example embodiment. In FIG. 16, the same steps as those illustrated in FIG. 3 carry the same reference numerals.

As illustrated in FIG. 16, when the operation of the biometric authentication system 10 according to the sixth example embodiment is started, first, the acquisition unit 110 obtains the feature quantity of the living body (step S11). Subsequently, the generation unit 120 generates a plurality of divided feature quantities by dividing the feature quantity (step S12). Subsequently, the collation unit 130 collates the divided feature quantities by using the threshold for each living body (i.e., the threshold stored in the individual threshold storage unit 131) (step S71).

### (Technical Effect)

Next, a technical effect obtained by the biometric authentication system 10 according to the sixth example embodiment will be described.

As described in FIGS. 15 and 16, in the biometric authentication system 10 according to the sixth example embodiment, the collation process is performed by using the threshold that is different for each living body. In this way, an appropriate threshold can be used in accordance with the living body to be collated, and it is therefore possible to obtain more appropriate result than those when the same threshold is used for all the living bodies.

### Seventh Example Embodiment

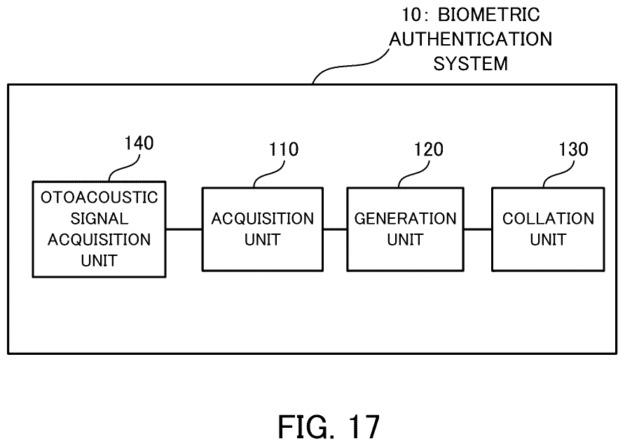

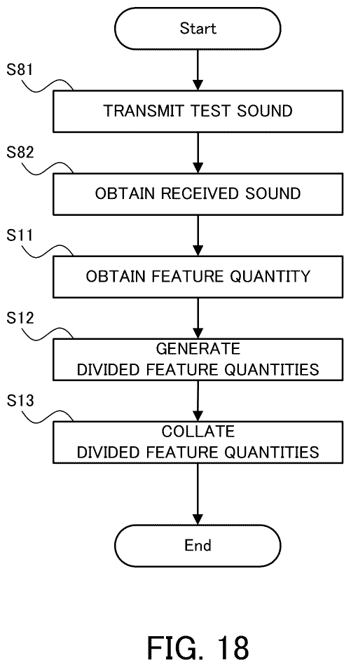

The biometric authentication system 10 according to the seventh example embodiment will be described with reference to FIG. 17 and FIG. 18. The seventh example embodiment is partially different from the first to sixth example embodiments only in the configuration and operation, and may be the same as the first to sixth example embodiments in the other parts. For this reason, a part that is different from each of the example embodiment described above will be described in detail below, and a description of the other overlapping parts will be omitted as appropriate.

### (Functional Configuration)

First, with reference to FIG. 17, a functional configuration of the biometric authentication system 10 according to the seventh example embodiment will be described. FIG. 17 is a block diagram illustrating the functional configuration of the biometric authentication system according to the seventh example embodiment. In FIG. 17, the same components as those illustrated in FIG. 2 carry the same reference numerals.

As illustrated in FIG. 17, the biometric authentication system 10 according to the seventh example embodiment includes, as processing blocks for realizing its functions, the acquisition unit 110, the generation unit 120, the collation unit 130, and an otoacoustic signal acquisition unit 140. That is, the biometric authentication system 10 according to the seventh example embodiment further includes the otoacoustic signal acquisition unit 140 in addition to the components in the first example embodiment (see FIG. 2).

The otoacoustic signal acquisition unit 140 is configured as a device for performing otoacoustic authentication, for example. Specifically, the otoacoustic signal acquisition unit 140 is configured as a device capable of transmitting a test sound to an ear hole of the living body and receiving a received sound that is received as a consequence that the test sound is reflected in the ear hole. A more detailed description of the otoacoustic authentication will be omitted here, because the existing techniques/technologies can be adopted as appropriate. Information about the received sound that is received by the otoacoustic signal acquisition unit is configured to be outputted to the acquisition unit 110.

### (Flow of Operation)

Next, with reference to FIG. 18, a flow of operation of the biometric authentication system according to the seventh example embodiment will be described. FIG. 18 is a flowchart illustrating the flow of the operation of the biometric authentication system according to the seventh example embodiment. In FIG. 18, the same steps as those illustrated in FIG. 3 carry the same reference numerals.

As illustrated in FIG. 18, when the operation of the biometric authentication system 10 according to the seventh example embodiment is started, first, the otoacoustic signal acquisition unit 140 transmits the test sound to the ear hole of the living body (step S81). The otoacoustic signal acquisition unit 140 then obtains information about the received sound that is received as a consequence that the test sound is reflected in the ear hole (step S82). Subsequently, the acquisition unit 110 obtains the feature quantity of the living body from the information obtained by the otoacoustic signal acquisition unit 140 (step S11). Subsequently, the generation unit 120 generates a plurality of divided feature quantities by dividing the feature quantity (step S12). Then, the collation unit 130 performs the collation process of the living body by using the divided feature quantities (step S13).

### (Technical Effect)

Next, a technical effect obtained by the biometric authentication system 10 according to the seventh example embodiment will be described.

As described in FIG. 17 and FIG. 18, in the biometric authentication system 10 according to the seventh example embodiment, the feature quantity of the living body is obtained by the otoacoustic authentication. Then, a plurality of divided feature quantities are generated from the feature quantity, and the collation process is performed. In this way, it is possible to improve an authentication accuracy of the otoacoustic authentication. For example, in the collation of the feature quantity of the otoacoustic authentication, a process that uses machine-learning to modify the feature quantity is sometimes performed in order to improve the authentication accuracy. If the division of the feature quantity in the example embodiment is applied after such a modification process, the accuracy of the otoacoustic authentication can be further improved. In addition, even by using the machine-learning to perform a different modification process on a plurality of divided feature quantities, the accuracy of the otoacoustic authentication can be improved as well.

As a way to improve the accuracy of the biometric authentication, machine-learning that uses personal data on the person in question labeled as teacher data and data on another person is considered. In this case, the data on another person is required. In the biometric authentication system 10 in each of the above-described example embodiments, however, the feature quantity of one person can be divided into a plurality of divided feature quantities, and thus, the accuracy of the biometric authentication can be improved only by the personal data.

### <Supplementary Notes>

The example embodiments described above may be further described as, but not limited to, the following Supplementary Notes below.

### (Supplementary Note 1)

A biometric authentication system described in Supplementary Note 1 is a biometric authentication system including: an acquisition unit that obtains a feature quantity of a living body; a generation unit that divides the feature quantity and generates a plurality of divided feature quantities; and a collation unit that performs a collation process of the living body by using the plurality of divided feature quantities.

### (Supplementary Note 2)

A biometric authentication system described in Supplementary Note 2 is the biometric authentication system described in Supplementary Note 1, wherein the generation unit divides the feature quantity into a plurality of values and combines some of the plurality of values to generate the plurality of divided feature quantities.

### (Supplementary Note 3)

A biometric authentication system described in Supplementary Note 3 is the biometric authentication system described in Supplementary Note 2, wherein the generation unit combines some of the plurality of values, with each combination including the same number of values, and generates the plurality of divided feature quantities.

### (Supplementary Note 4)

A biometric authentication system described in Supplementary Note 4 is the biometric authentication system described in Supplementary Note 2 or 3, wherein the generation unit combines some of the plurality of values so as to redundantly include the plurality of values in the plurality of divided feature quantities, and generates the plurality of divided feature quantities.

### (Supplementary Note 5)

A biometric authentication system described in Supplementary Note 5 is the biometric authentication system described in any one of Supplementary Notes 1 to 4, wherein the collation process is a process of calculating a plurality of matching scores as a collation result for each of the plurality of divided feature quantities, and of outputting a result obtained by integrating the plurality of matching scores.

### (Supplementary Note 6)

A biometric authentication system described in Supplementary Note 6 is the biometric authentication system described in any one of Supplementary Notes 1 to 5, wherein the collation unit performs the collation process by using a threshold that varies for each living body.

### (Supplementary Note 7)

A biometric authentication system described in Supplementary Note 7 is the biometric authentication system described in any one of Supplementary Notes 1 to 6, wherein the acquisition unit transmits a test sound to an ear hole of the living body and obtains the feature quantity on the basis of a received sound that is received as a consequence that the test sound is reflected in the ear hole.

### (Supplementary Note 8)

A biometric authentication method described in Supplementary Note 8 is a biometric authentication method including: obtaining a feature quantity of a living body; dividing the feature quantity and generating a plurality of divided feature quantities; and performing a collation process of the living body by using the plurality of divided feature quantities.

### (Supplementary Note 9)

A computer program described in Supplementary Note 9 is a computer program that operates a computer: to obtain a feature quantity of a living body; to divide the feature quantity and generate a plurality of divided feature quantities; and to perform a collation process of the living body by using the plurality of divided feature quantities.

### (Supplementary Note 10)

A recording medium described in Supplementary Note 10 is a recording medium on which the computer program described in Supplementary Note 9 is recorded.

This disclosure is not limited to the examples described above and is allowed to be changed, if desired, without departing from the essence or spirit of this disclosure which can be read from the claims and the entire specification. A biometric authentication system, a biometric authentication method, and a computer program with such changes are also intended to be within the technical scope of this disclosure.

### DESCRIPTION OF REFERENCE CODES

10 Biometric authentication system
11 Processor
14 Storage apparatus
110 Acquisition unit
120 Generation unit
121 Group setting unit
130 Collation unit
131 Individual threshold storage unit
140 Otoacoustic signal acquisition unit

What is claimed is:

1. A biometric authentication system comprising:
at least one memory that is configured to store instructions; and
at least one first processor that is configured to execute the instructions to
obtain a feature quantity of a living body;
divide the feature quantity and generate a plurality of divided feature quantities; and
perform a collation process of the living body by using the plurality of divided feature quantities.

2. The biometric authentication system according to claim 1, wherein the at least one first processor is configured to execute the instructions to divide the feature quantity into a plurality of values and combine some of the plurality of values to generate the plurality of divided feature quantities.

3. The biometric authentication system according to claim 2, wherein the at least one first processor is configured to execute the instructions to combine some of the plurality of values, with each combination including the same number of values, and generate the plurality of divided feature quantities.

4. The biometric authentication system according to claim 2, wherein at least one first processor is configured to execute the instructions to combine some of the plurality of values so as to redundantly include the plurality of values in the plurality of divided feature quantities, and generate the plurality of divided feature quantities.

5. The biometric authentication system according to claim 1, wherein the collation process is a process of calculating a plurality of matching scores as a collation result for each of the plurality of divided feature quantities, and of outputting a result obtained by integrating the plurality of matching scores.

6. The biometric authentication system according to claim 1, wherein the at least one first processor is configured to execute the instructions to perform the collation process by using a threshold that varies for each living body.

7. The biometric authentication system according to claim 1, wherein the at least one first processor is configured to execute the instructions to transmit a test sound to an ear hole of the living body and obtains the feature quantity on the basis of a received sound that is received as a consequence that the test sound is reflected in the ear hole.

8. A biometric authentication method comprising:
obtaining a feature quantity of a living body;
dividing the feature quantity and generating a plurality of divided feature quantities; and
performing a collation process of the living body by using the plurality of divided feature quantities.

9. A non-transitory recording medium on which a computer program that allows a computer to execute a biometric authentication method is recorded, the biometric authentication method including:
obtaining a feature quantity of a living body;
dividing the feature quantity and generating a plurality of divided feature quantities; and
performing a collation process of the living body by using the plurality of divided feature quantities.
